(19) United States
(12) Patent Application Publication
(10) Pub. No.: US 2024/0416552 A1
(43) Pub. Date: Dec. 19, 2024

(54) DEVICE AND METHOD FOR DETECTION OF FOREIGN OBJECTS IN A MAT SPREAD ON A CONVEYOR

(71) Applicant: Siempelkamp Maschinen- und Anlagenbau GmbH, Krefeld (DE)

(72) Inventors: Rainer Krumbach-Voß, Tönisvorst (DE); Pedro Robles Malagrino, Krefeld (DE); Klaus Schürmann, Jüchen (DE); Günter Staub, Krefeld (DE)

(73) Assignee: Siempelkamp Maschinen- und Anlagenbau GmbH, Krefeld (DE)

(21) Appl. No.: 18/742,521

(22) Filed: Jun. 13, 2024

(30) Foreign Application Priority Data
Jun. 13, 2023 (DE) 10 2023 002 398.1

(51) Int. Cl. B27N 3/18; B27N 3/16

(52) U.S. Cl. CPC B27N 3/18; B27N 3/16

(57) ABSTRACT

A method for detecting at least one foreign object in a mat includes: providing that the mat is spread on a conveyor device in a process for producing manufactured boards, the mat including (i) fibers or chips and (ii) at least one binding agent which are pressed under an increased temperature in a continuous press; using a first sensor device, which operates with electromagnetic waves, ultrasonic waves, magnetic fields, or radiometry and which is directed at at least one spatial measuring area of the mat in order to detect the foreign object, the first sensor device recognizing a material of the foreign object; aligning—if necessary, temporally offset—a second sensor device with the at least one spatial measuring area; and determining, by the second sensor device, at least one foreign object dimension of the foreign object.

### CROSS REFERENCE TO RELATED APPLICATIONS

This claims priority to German patent application 10 2023 002 398.1, filed Jun. 13, 2023, which is incorporated herein by reference.

### BACKGROUND OF THE INVENTION

### 1. Field of the Invention

The present invention relates to detection of foreign objects in a mat on a conveyor device.

### 2. Description of the Related Art

Manufactured boards are primarily understood to be wood-based panels that are already produced, optionally in a continuous press, for example, chip-, MDF or OSB boards, wherein the particles contained in the mat are then essentially wood fibers or wood chips that have been provided with a binding agent. In more recent times, other lignocellulosic materials, for example the fibers of annual plants (for example rice straw), have also been used to form manufactured boards, by way of

The fibers or chips are spread in a spreading station onto a conveyor system, usually on a conveyor belt, also known as a forming belt, where they form a mat that is conveyed in the direction of travel of the press. In the continuous press, hydraulic components apply high pressure via mostly heated pressure plates and rolling elements to the upper and/or lower steel press belt, between which the mat is compacted at the same feed rate as that of the press belts. Said press belts are generally only 0.5 to 4 mm thick and are overly sensitive to excessive punctiform pressures. Excessive denting or damage can be the reason why only rejects are produced on the entire system. Changing the press belts is cost- and labor-intensive and understandably undesirable. Considering that in the continuous press compactions and thus also changes in thickness occur—that is, from the mat to the manufactured board—it is apparent that even relatively small hard foreign objects inside the mat can already push through the surface and damage the press belts. Typical compaction values in kg/m³ are:

| Product | Density after spreading | Density after press |
|---|---|---|
| MDF | 18-35 | 600-900 |
| Chip | 140-150 | 550-750 |
| OSB | 60-70 | 500-700 |

In particular in the case of MDF boards, the thickness of the mat relative to the manufactured board is reduced to such an extent that even smaller trapped foreign objects can easily push through the surface.

However, chips or fibers are subjected to various processes prior to spreading, such as shredding, washing or spraying with binding agents until they are stored in large bin in front of or above the spreading station. Often, the multiple upstream processes result in unwanted foreign objects being integrated into the spreading process and remaining in the mat. Depending on the size and hardness of the foreign object, these can protrude from the surface during the compacting process in the press and cause damage to the press belts.

It is therefore customary to check the mat before entering the press. For example, a metal detector is often used for this purpose. However, systems are also known where the web is X-rayed. If a foreign object is found, the manufacturing process is stopped by removing the affected part of the mat by way of a foreign object removal device. This can be a type of drop chute that opens when the conveyor belt is pulled back slightly. But there are also systems known where the contaminated mat part is vacuumed or brushed off the conveyor belt.

In DE 10 2004 048 275 B3, for example, the possible use of a metal detector is described. Such metal detectors work inductively with a transmitter coil through which an alternating current flows, which causes an alternating magnetic field. If this alternating field penetrates a conductive material positioned underneath the probe, eddy currents are induced there, which in turn generate a measurable magnetic field. Metal parts pose the highest danger due to their density and metal detectors are relatively inexpensive. The detector can detect individual metal parts accurately enough. However, non-ferrous metals such as copper or aluminum are not included.

However, metal parts are also greatly reduced in size during production runs (chips, dust). A metal detector integrates the metal mass into its field range and cannot distinguish between a single larger solid object and numerous small ones. This means that the metal detector will also detect a harmless area of metal dust as metal foreign objects and thus interrupt production unnecessarily.

On the other hand, the metal detector cannot locate harmful larger glue clumps or, for example, a plastic nut.

Other arrangements for foreign object detection have similar problems. A sensor device based on X-rays with appropriate area resolution can also detect the silhouette of an object with correspondingly higher densities. However, the scanner cannot detect the height and thus the volume and mass of the object. Thus, not only metallic foreign objects are recognizable, but also those consisting of other materials with a correspondingly high density.

However, since the foreign object scanner can only detect the surface of the foreign object, a flat object that is harmless to the press belt would result in a false triggering of the foreign object removal device.

In addition to the inductive metal detector and the X-ray scanner, there are numerous other theoretical sensor devices for detecting foreign objects in a mat. These include:
a) Image sensors with visible and invisible light;
b) Ultrasonic transmitters and sensors;
c) Sensors according to the magneto-optical Kerr-effect;
d) Radioactive or radiometric sensors;
e) Magnetic sensors (MRI).

These possible sensors and measurement methods all have in common that they cannot capture the foreign object in its entirety and would often cause unnecessary production interruptions in order to protect the press belt.

What is needed in the art is to be able to better evaluate in the manufacturing process of manufactured boards when a foreign object could damage the steel belt of a continuous press and thus raise the threshold for triggering a foreign object removal and consequently to interrupt production less frequently.

### SUMMARY OF THE INVENTION

The present invention relates to a device for the detection of foreign objects in a mat spread on a conveyor device in the process of manufactured board production, wherein the mat consists at least partially of fibers or chips and binding agents, which are pressed under increased temperature and wherein at least one first sensor device is provided which operates with electromagnetic or ultrasonic waves or with magnetic fields or with radiometry and which is directed at one or more spatial measuring areas of the mat in order to detect foreign objects.

The present invention also relates to a method for detecting foreign objects in a mat spread on a conveyor device in the process of manufactured board production, wherein the mat consists at least partially of fibers or chips and binding agents, which are pressed under t increased temperature, wherein at least one sensor device is provided which operates with electromagnetic or ultrasonic waves or with magnetic fields or with radiometry and which is directed at one or more spatial measuring areas of the mat in order to detect foreign objects.

The present invention further relates to a production line for the production of manufactured boards having a spreading station for spreading a mat on a conveyor device, wherein the mat consists at least partially of fibers or chips and binding agents, and a press for compacting the mat, which includes the device according to the present invention for detecting foreign objects, as well as a manufacturing process for manufactured boards including a spreading station, wherein a mat is spread onto a conveyor device, wherein the mat consists at least partially of fibers or chips and binding agents and is compacted in a press, using the method according to the present invention of detecting foreign objects.

As indicated above, manufactured boards are primarily understood to be wood-based panels that are already produced, optionally in a continuous press, for example, chip-, MDF or OSB boards, wherein the particles contained in the mat are then essentially wood fibers or wood chips that have been provided with a binding agent. In more recent times, other lignocellulosic materials, for example the fibers of annual plants (for example rice straw), have also been used to form manufactured boards. According to the present invention, manufactured boards can also be insulation panels, gypsum panels, plastic panels, or other manufactured boards whose particles have been pressed by way of revolving belts in a continuous press with heat treatment. Such a press is marketed by the applicant under the name ContiRoll. In this process, binding agents are usually added to the particles before the continuous press so that the manufactured boards obtain a high degree of stability.

As indicated above, in addition to the inductive metal detector and the X-ray scanner, there are numerous other theoretical sensor devices for detecting foreign objects in a mat. These include:
a) Image sensors with visible and invisible light;
b) Ultrasonic transmitters and sensors;
c) Sensors according to the magneto-optical Kerr-effect;
d) Radioactive or radiometric sensors;
e) Magnetic sensors (MRI).
   All these possible sensors and measurement methods may be part of the present invention and be present in the first sensor device or procedurally represent its mode of operation. However, they all have in common that they cannot capture the foreign object in its entirety and would often cause unnecessary production interruptions in order to protect the press belt.

The present invention provides a device for the detection of foreign objects in a mat spread on a conveyor device in the process of manufactured board production, wherein the mat consists at least partially of fibers or chips and binding agents, which are pressed under increased temperature, wherein at least one first sensor device is provided which operates with electromagnetic or ultrasonic waves or with magnetic fields or with radiometry and which is directed at one or more spatial measuring areas of the mat in order to detect foreign objects, characterized in that at least one second sensor device is provided multimodally, which is also aligned with measuring areas, and wherein first sensor device is suitable for performing a material recognition of the foreign object, and the second sensor device is suitable for determining at least one foreign object dimension.

The present invention also provides a method for the detection of foreign objects in a mat spread on a conveyor device in the process of manufactured board production, wherein the mat consists at least partially of fibers or chips and binding agents, which are pressed under increased temperature in a continuous press, wherein at least one first sensor device is used which operates with electromagnetic or ultrasonic waves or with magnetic fields or with radiometry and which is directed at one or more spatial measuring ranges of the mat in order to detect foreign objects, characterized in that at least one second sensor device is provided, which is also aligned—if necessary, temporally offset—with the measuring ranges, and wherein the first sensor device performs a material recognition of the foreign object, and the second sensor device determines at least one foreign object dimension. Thus, the method provides for using at least one second sensor device multimodally, which is also aligned with the measuring areas, possibly with a time delay, wherein the first sensor device is used to detect the material of the foreign object and the second sensor device determines at least one foreign object dimension.

With the combination of a first sensor device, which is suitable for at least approximate material detection of the foreign object and a second sensor device, which is suitable for determining the dimension of a foreign object, a detrimental foreign object can be identified clearly better than previously.

There may be systems wherein—in throughput direction of the mat—the first sensor device is used first and only then the second sensor device is used for detection. In other systems, exactly the opposite order may make more sense. This depends on the spread material and the thickness or density of the mat. In one case, it is optional to determine the material first and then the dimensions of the foreign object; in another case it may be more useful to determine the dimensions of the foreign object first and then the material. Thus, the terms “first” and “second” sensor device only express that there are two different sensor devices that can follow each other in any desired sequence.

The term “measuring area” refers to the area or spatially limited areas that the respective sensor device can detect. Such a measuring area can extend over the entire width and/or thickness of the mat.

Optionally, the first sensor device has a sensor to detect the metal content of a foreign object. With such a sensor, for example a detector supported by electromagnetic coils, that is, inductively, one is informed immediately if there is a ferrous metal part in the mat. In combination with an also optional second sensor device, which is suitable for detecting the dimensions of the foreign object material optionally three-dimensionally (sensors supported by X-rays or radiometry are herein suitable, for example), the hazard that the foreign object poses for the press belt can be determined. For example, the operator of the production line knows immediately that he does not have to interrupt the production of manufactured boards if the metal detector has reacted, but the second sensor device gives the signal that the issue concerns only scattered tiny metal particles.

However, it is also optional that the first sensor is equipped with or connected to a detection unit to measure a phase shift of the signal, caused by a detected metal particle, in order to thereby enable identification of the type of metal and thereby to be able to distinguish between ferrous and non-ferrous metals. In particular, this makes it possible to identify the type of metal. A distinction can be made between copper, brass, aluminum, iron and steel, and even between different types or categories, for example between cast iron or structural steel, depending on the density.

An evaluation device that is able to perform a sensor fusion of at least one first sensor device and at least a second sensor device is provided particularly advantageously.

Sensor fusion algorithmically combines the measurement results of the at least one first sensor device and the at least second sensor device and thus creates a complete and precise evaluation of the state of the foreign object. For example, it then uses a predefined harmfulness matrix to determine whether the foreign object is harmful to the press belt or not. The matrix provides a clear classification depending on foreign object material and foreign object dimensions, for example. In the same way, dependency algorithms can be stored with formulas that define a critical state of the foreign objects when a calculated threshold value is exceeded.

Optionally, in a case of assessed possible harmfulness to the press belt, that is, depending on the result of the sensor fusion in the evaluation device, a foreign object removal device can be activated.

Opening of a removal drop chute or activation of an extraction device by this inventive device for the detection of foreign objects is therefore completely automatic. The operator of the production line can also be sure that the production interruption will only take place if foreign objects are detected that can potentially damage the press belt or at least if there is a high probability of such damage occurring. According to the evaluation device, for example, in the case where a large but very flat foreign object is hidden in the mat, which would already cause a production stop with a conventional foreign object scanner, it would be possible to continue running the production line and, if necessary, to cut the section containing the foreign object out of the manufactured board that will be created later. As a result, the production line does not have to be stopped for foreign objects that are detected but are harmless to the press belt.

The tendency in the production of manufactured boards is to be able to produce ever thinner and lighter panels. These are particularly affected by the problem, and the device according to the present invention is particularly suitable for mat thicknesses <8 mm. It is therefore advantageous if, in addition to at least one first sensor device and the at least one second sensor device, a thickness, density or basis weight measuring device for the mat is provided.

Such thickness, density or basis weight measuring devices are common in the production of spread mats that are pressed into manufactured boards and are known to experts. As a rule, however, they are only used to measure the uniformity over the spreading height and/or over the width and along the conveyor belt.

In the case of the device according to the present invention, however, the determined thickness or density or basis weight measurement can also be transmitted to the evaluation unit. This measured value can also be considered in the evaluation matrix for the foreign object material and the foreign object dimension. In the case of thin mats, even smaller foreign objects can endanger the press belt.

It is particularly advantageous that a distance/time detection device is provided for the synchronization of the sensor signals of the at least one first sensor device and the at least one second sensor device.

In particular, if the at least one first sensor device and the at least one second sensor device are located at a distance from one another along the travel path of the mat, it must be ensured that the measured values of the first sensor device and the measured values of the second sensor device from the same measuring location are assigned to each other. Therefore, the measured values of the measuring location downstream in the direction of transport arrive at the evaluation device later, depending on the transport speed of the mat. This temporal offset must be considered. With the distance/time detection device, it is possible to record this time difference and pass it on to the evaluation unit. In many cases, the distance of the sensor device is fixed, so that the distance/time detection device only transmits the speed of the conveyor device, for example the conveyor belt. The evaluation device can then calculate the temporal offset of the measurement signals and take this into account when analyzing the foreign objects.

Alternatively, it may also be provided that the at least one first sensor device and the at least one second sensor device simultaneously detect the same measuring point on or in the mat. In this case, they could even be accommodated in a housing.

It may also be advantageously provided that the evaluation device is designed for damage history analysis.

This can result in considerable synergetic effects. The device has at least one first sensor device which is directed at one or more spatial measuring areas of the mat to recognize foreign objects, and has at least one second sensor device which is also directed towards the measuring areas, and wherein the first sensor device is suitable for performing a material detection of a foreign object, and the second sensor device is suitable for determining at least one dimension of a foreign object. This results in that, if the evaluation device is designed in such a way that a damage history analysis can be carried out, the possibility arises that data that can be obtained in particular from a sensor fusion and, in particular, that data obtained from a sensor fusion can be used as indications of a damage history analysis that is to be performed and can be made available and, in particular, is made available. Overall, this results in the advantage that a usable degree of informative value of the damage assessment is achievable, which, in many cases, is already used in practice. If foreign objects of a certain size and material are detected, this can already indicate certain damage in production or in the devices and/or equipment used in production. In this regard, it can be particularly informative if, in addition to the size, the shapes of the foreign objects are detected, especially three-dimensionally. It may therein be provided that the sensor technology of the evaluation device provides the shape in sufficient resolution to allow an edge observation of the shape of the foreign object that is to be observed, which allows further conclusions to be drawn about possible damage within the damage history analysis, in particular within the device(s) and/or equipment used for production. Likewise, patterns in the spatial and/or temporal distribution of the detection can be processed within the damage history analysis in order to further increase the degree of informative value. For example, the damage history analysis can be designed in such a way that pulsating patterns of foreign objects can be assigned and for example grouped according to different edge shapes, even if the detected foreign objects consist of the same material and are about the same size; they can therefore be assigned within the damage history analysis unit to a different (sub-) group from which a different group of possible causes of damage is inferred. The damage history analysis (unit) is therefore optionally also designed for group and/or matrix formation.

In addition, or alternatively, it can also be greatly advantageous if the device is further developed in such a way that the evaluation device is designed to predict damage.

Analogous further developments may also provide for interaction with a damage prediction unit which can predict damage from the data obtained to a certain degree of informative value. However, special synergies also arise here if the evaluation device is equipped with a damage history analysis (unit) and a damage prediction unit or is operatively connected with them and if the two analyses or analysis units are also networked with each other.

The evaluation device can then both recognize an image of damage from the signals (data) obtained via the at least two sensors and, in particular, assign it with a particularly high degree of informative value to a special device (station) of a production line for manufactured boards and predict when the damage will have reached a certain level in the future. The operator of such a production line can then determine a measure by which the line will be shut down for maintenance in order to avoid a much more costly crash. The operator can then also decide—if informed in good time—whether he will replace a detected wear-causing component early within a planned maintenance procedure.

Considerable advantages result, especially in connection with one or both of the aforementioned design variants, if the evaluation device includes an adaptive unit or is operatively connected with an adaptive unit.

In this manner, continuous improvement of the detection and analysis competence of the device is possible, and ever improved levels of informative values are achievable. Above all, however, the provision of an adaptive unit in the context described herein is advantageous because formulations often have to be changed in the production of manufactured boards. There are essentially two reasons for this: on the one hand, not all the necessary raw materials are always available in the same quality and at the same price for the production of a certain type and grade of manufactured board, so that the operator of a line for the production of manufactured boards usually has to adapt the formulation of his manufactured board quite frequently over time for economic or technical reasons, sometimes even daily. On the other hand, the purchase of a production line for producing manufactured boards is associated with high costs in the two- or even in the low three-digit million Euro range. As a result, the production line must be operated for many years, usually for at least two to three decades, often even longer. However, since the requirements of the market can change considerably over such a time period, the production line must also be designed in such a way that it is possible for the operator to produce other grades of manufactured boards, for example chip board panels, for example thin panels, medium-thickness panels, heavy thickness panels in a single or multi-layer design, or even different types of manufactured boards, for example, chipboard panels, hybrid panels such as chipboard panels with MDF top layers or similar.

The adaptive unit can then help to transfer knowledge gained from the manufacturing process of one grade of one type to another grade of this type or even to the manufacturing process of another type of manufactured boards, especially within one and the same production line. The detectable foreign objects and foreign object patterns do not necessarily have to originate from line or device and/or station of the production line, but can also be generated from the manufacturing process, especially if they are clumps or if foreign object particles were present in a raw material. This can be particularly the case when processing recycled material. In addition, annual plants, especially grass-like plants, often have high silicate contents. Although these can often be separated quite well from the useful components (fiber content) in the preparation process, for example by way of area of shredding, defibration and/or/or cooking, they cannot always be completely removed from the mass flow in the manufacturing process. Here, too, unwanted or even dangerous accumulations can occur in the mat.

For the further development of the aforementioned embodiment, it is of particular advantage if the adaptive unit is designed to identify at least possible damage images on the basis of patterns of detected signals from the individual sensors.

Thus, the added value of such a device is again significantly increased, since it was surprisingly shown in tests that the use of such a device in connection with the production of manufactured boards can lead to unforeseeable reductions in analysis times. It is not only important to achieve a high degree of informative value, but it can also be of great importance to receive orientation regarding the situation in a timely manner. For example, the adaptive unit can assign pulsating patterns to revolving processes or units; the length of the revolution can then be deduced from the repetition interval. If different revolving belts (for example steel belts) are used in a process or in a system designed to carry out a process, for example a system for the production of manufactured boards, the repetition interval can be used to limit or assign the damaged unit (station).

Different raw materials can also tend to form different damage patterns and can be used as indicators if the adaptive unit is trained accordingly.

In addition, there are great advantages if the device is operatively connected with at least one sensor that is assigned to a device/station of a production line for the production of manufactured boards.

It is thus possible to obtain a closed informational image. The evaluation unit can compare foreign objects detected by at least two sensors and, in particular, patterns of foreign objects arising over a certain period of time with signals from the causative and/or processing units (stations) of a (production) line for the production of manufactured boards. It is particularly valuable if the evaluation unit is designed for sensor fusion with at least one other sensor, in particular at least one sensor assigned to a device/station of a production line for the production of manufactured boards.

Procedurally, the advantages are analogous to those described in regard to the device. The claims pertaining to the method essentially correspond to the claims pertaining to the device for the detection of foreign objects in a mat spread on a conveyor device.

It is significant that there are also clear advantages in the method compared to the state of the art. Assuming, for example, a metal detector as the first sensor device and a second sensor device that detects the dimensions of the foreign object material in three dimensions, the following three examples show how effective sensor fusion can be:
The second sensor device detects a relevant area or dimension of an object. The metal mass consistent with the object surface is detected by way of the first sensor device. This indicates the likely presence of a correspondingly hazardous metal object, and a foreign object removal is activated.
The second sensor device does not detect any suspicious area or dimension, but the metal detector reports a relevant metal mass. This indicates a larger field of distributed metal dust and no hazardous foreign objects. Foreign object removal can be omitted.
The second sensor device detects a relevant area or dimension of an object. No metal is detected by way of the first sensor device. This indicates the likely presence of a non-metallic object (for example, a lump of glue) that would be treated accordingly if it were above a separate threshold. Beyond a certain dimension the value of which also depends on the thickness of the mat and the press nip height, a foreign object removal is activated.

It is advantageous in a production line to use both the inventive device and the analogous and corresponding method of the present invention for detection of foreign objects between the spreading station and the continuous presses. Thus, the present invention also provides a production line for producing manufactured boards having a spreading station for spreading a mat on a conveyor device, wherein the mat consists at least partially of fibers or chips and binding agents, and a continuous press for compacting the mat, characterized in that, between the spreading station and the continuous press a device is provided for detection of foreign objects, the device being described above. Further, the present invention also provides a manufacturing process for the production of manufactured boards, including a spreading station in which a mat is spread on a conveyor device, wherein the mat consists at least partially of fibers or chips and binding agents and is compacted in a continuous press, characterized in that between the spreading station and the continuous press a method for detection of foreign objects is applied, the method being described above.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a schematic side view of a system for the production of a manufactured board in the concept of the present invention, with a device according to the present invention for the detection of foreign objects;

FIG. 2 is an alternative arrangement of the sensor devices; and

FIG. 3 is an additional embodiment according to the present invention.

Corresponding reference characters indicate corresponding parts throughout the several views. The exemplifications set out herein illustrate embodiments of the invention, and such exemplifications are not to be construed as limiting the scope of the invention in any manner.

### DETAILED DESCRIPTION OF THE INVENTION

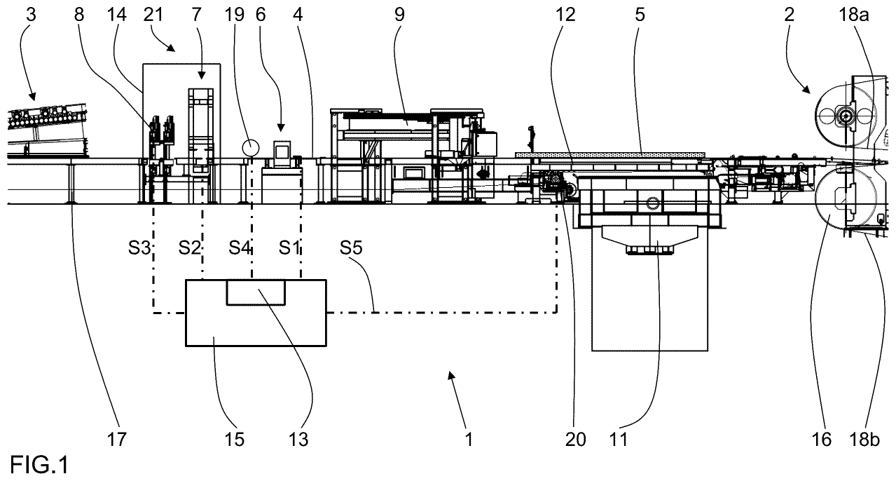

FIG. 1 schematically shows a section of a system 1 for the production of a manufactured board.

On the left side, the end of a spreading station 3 is indicated, with which chips or fibers wetted with binding agents are spread onto a long conveying device 4, in this embodiment a conveyor belt running over a support structure 17, to form a mat 5. Mat 5 can have several different layers of chips or fibers of different sizes, which may also be spread, oriented in different directions. For reasons of clarity, only a short section of mat 5 is schematically shown in FIG. 1.

On the right edge, that is, the end of the conveyor section, is the infeed into a continuous press 2. It has an upper press belt 18a and a lower press belt 18b, which continuously revolve around various deflection rollers 16 and can apply pressure to mat 5 from both sides. Press belts 18a, 18b are thin metal strips that can be affected by selective pressure caused by sharp or hard foreign objects in such a way as to transfer damage to compressed mat 5 and is visible on the manufactured board on discharge from press 2 (not shown).

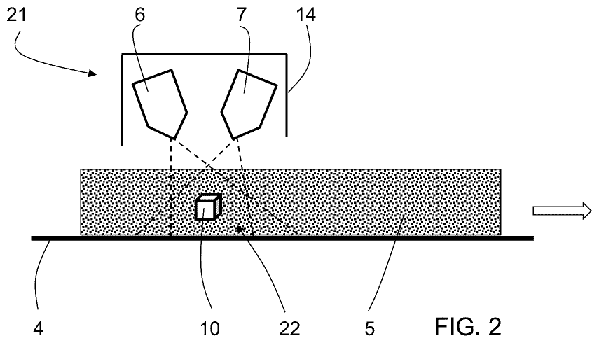

This means that a foreign object 10 in mat 5 (shown in more detail in FIG. 2) which endangers press conveyor belt 18a, 18b must be removed by way of a foreign object removal device 11 before it enters continuous press 2. In the design example, foreign object removal device 11 is a type of drop chute that can be opened by pulling back a cover and/or a movable conveyor belt section 12 via adjustment motors 20. After removing the mat section, which contains a foreign object 10 that potentially endangers press belts 18a, 18b, mat 5 no longer has a straight edge. For this reason, line 1 for the production of a manufactured board is equipped with a diagonal saw 9, which can saw a new straight edge into mat 5 at right angles to the feed direction. This edge can enter continuous press 2 without problems.

According to the present invention, the new feature of this system is device 21 and the method for detecting foreign objects in a mat 5 that is spread onto conveyor device 4. The decisive factor is that a first sensor device 6 and a second sensor device 7 are used. First sensor device 6 is suitable for material detection of the foreign object, and second sensor device 7 is suitable for determining the dimensions of the foreign object. In this embodiment, sensor device 7 is preceded by sensor device 6. In other cases, the two sensor devices 6, 7 work in reverse order, as is also indicated schematically in FIG. 2.

Referring again to FIG. 1: The physical measuring system of sensor device 7 is equipped to allow it to determine the dimensions of a foreign object, if necessary, even three-dimensionally. In this design example, it may for example be in the embodiment of a sensor device (7) that acts with X-rays. In further progression of the mat, the same spatial measuring area 22, in which a foreign object 10 (see also FIG. 2) of larger dimensions was detected, is examined by sensor device 6. Sensor device 6 can determine the material of the foreign object 10 or at least its density, so that based on the dimensions and the material characteristics a statement can be made as to whether foreign object 10 is harmful to press belts 18a, 18b in continuous press 2. It may herein be sufficient to determine the metal content in foreign object 10, which is possible with an inductive metal detector.

To aid in faster decision-making, evaluation device 15 is provided, which includes signal lines S1 to S5. It receives measured values from first sensor device 6 via signal line S1. Via signal line S2, it receives measured values from second sensor device 7. Since the foreign object threshold values stored in evaluation device 15 are strongly dependent on the mat thickness or density currently being run, the relevant data is supplemented by an additionally installed thickness-, density- or basis weight measuring device 8 via a signal line S3. To be able to consider the temporal distance of a measuring area 22 from second sensor device 7 to first sensor device 6 during measurements, evaluation device 15 also receives information from a conveyor belt speedometer 19 with signal line S4 via a displacement/time detection device 13.

If evaluation unit 15 detects that a foreign object is threatening press belts 18a and 18b of continuous press 2, it activates foreign object removal device 11.

In FIG. 2, the two sensor devices 6, 7 are arranged in a sequence converse to that in FIG. 1. However, sensor devices 6, 7 are accommodated in a housing 14 and are therefore arranged so close to each other that measuring areas 22 of sensor devices 6, 7 overlap at least partially and they both can perform their measurements simultaneously. Mat 5, which is transported on conveyor belt 4, contains a large foreign object 10. Keeping with the same sensor types as shown in the embodiment of FIG. 1, sensor device 6 recognizes the material, for example metal, and sensor device 7 identifies the dimensions, possibly even three-dimensionally, so that the connected evaluation device can immediately decide whether a foreign object rejection device needs to be activated on the basis of the measured values and stored threshold values. However, the present invention is also intended to include other measurement methods of sensor devices 6, 7. These include sensor devices that work with electromagnetic or ultrasonic waves or with magnetic fields or radiometry.

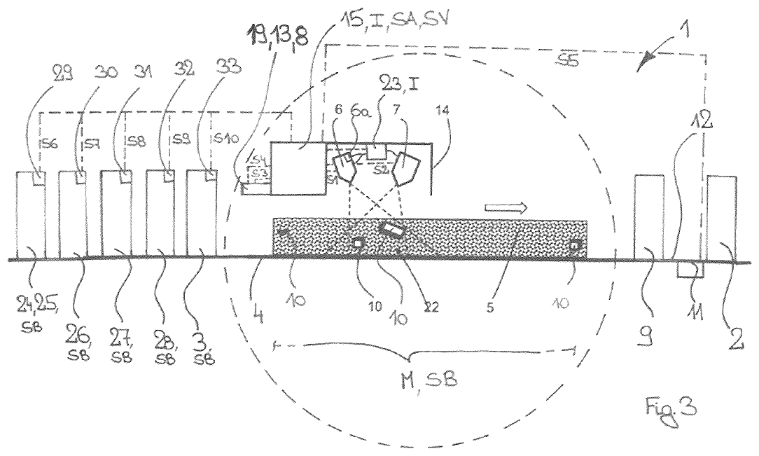

FIG. 3 shows an additional embodiment. Identical components and elements as in FIGS. 1 and 2 have the same identifications. The embodiments shown in the drawings are exemplary and interchangeable with each other in the context of the disclosure. FIG. 3 shows an embodiment of line 1 for the production of a manufactured board in a strongly schematized form. Most of the devices/stations are only shown as small boxes, wherein the shape and size of the boxes do not correspond to the allocation of the respective actual space requirements of the individual device/station in relation to another device/station. In the center of the drawing, important features relating to the present invention are illustrated in a circular area formed by broken lines in a highlighted, but also schematic representation. There it can be seen that the evaluation device 15 is designed for a damage history analysis SA.

This can give rise to considerable synergetic effects. Device 21 has at least one first sensor device 6 which is directed at one or more spatial measuring areas 22 of the mat to recognize foreign objects 10 and has at least one second sensor device 7 which is also directed towards measuring areas 22, and wherein first sensor device 6 is suitable for performing a material detection of foreign object 10, and second sensor device 7 is suitable for determining at least one dimension of a foreign object. This provides that, if evaluation device 15 is designed in such a way that a damage history analysis SA can be carried out, the possibility arises that data that can be obtained in particular from a sensor fusion and, in particular, that data obtained from a sensor fusion can be used as indications of a damage history analysis SA that is to be performed and can be made available and, in particular, is made available. It can be particularly informative if, in addition to the size, the shapes of the foreign objects are detected by second sensor 7, in particular three-dimensionally. For this reason, the sensor technology of evaluation device 15 provides the detected shape in sufficient resolution to allow an edge observation of the shape of the foreign object to be observed, which allows further conclusions to be drawn about possible damage images within damage history analysis SA, in particular within the device(s) used for production 24, 25, 26, 27, 28, 3, 4 and/or production line(s) 1. In addition, first sensor 6 has a detection unit 6a for measuring a phase shift of the signal generated by a detected metal particle, or is connected with the latter as shown, in order to enable identification I of the metal type, and thus to be able to distinguish between ferrous and non-ferrous metals, wherein sensor 6, with detection unit 6a, differentiates between copper, brass, aluminum, iron and steel, due to the densities even within the types into different categories, for example between cast iron or structural steel.

Likewise, patterns M can be processed in the spatial and/or temporal distribution of the detection within damage history analysis SA of evaluation device 15 in order to increase the degree of informative value. Damage history analysis SA is designed, for example, to assign pulsating patterns M of foreign objects 10 and to group them according to different edge shapes. Even if detected foreign objects 10 consist of the same material and are of about the same size, they can be assigned within damage history analysis device SA to another (sub-) group, from which a different group of possible causes of damage is inferred. In the example shown, damage history analysis (unit) SA is therefore also designed for group and/or matrix formation. In addition, in the embodiment shown, evaluation device 15 is also (at least optionally) designed for damage prediction SV, whereby the two analyses SA, SV or analysis units SA, SV are also networked with each other. In this way, it is particularly possible that evaluation device 15 recognizes a damage image SB from signals (data) S1, S2 obtained via the at least two sensors 6, 7 and, in particular, detect a damage image SB with a particularly high degree of informative value of a special device (station) 24, 25, 26, 27, 28, 3, 4 of a production line 1 for the production of manufactured boards, and predict when the damage will have reached a certain level in the future. The operator of such a production line 1 can then, for example, determine a measure at which production line 1 will be shut down for maintenance in order to avoid a much more costly crash. The operator can then also decide-if informed in a timely manner-whether he will replace a detected wear-causing early within a planned maintenance procedure.

Evaluation device 15 is also equipped with an adaptive unit 23 and, as demonstrated, is operatively connected with same. Adaptive unit 23 can even transfer knowledge gained from the manufacturing process of a first type of one type of manufactured board to be produced to another type of manufactured board to be produced or even to the manufacturing process of another type of manufactured board, in particular within the same line. Detectable foreign objects 10 and (foreign object) patterns M do not necessarily have to originate from production line 1 or from a device and/or station 24, 25, 26, 27, 28, 3, 4 of production line 1 but can also be generated within the manufacturing process, especially if they are clumps or foreign object particles that were present in a raw material. In order to manage this, adaptive unit 23 is designed to identify—at least possible—damage images SB on the basis of patterns M of detected signals S1 to S10 of individual sensors 6, 7, 8, 19, 29, 30, 31, 32, 33. For example, adaptive unit 23 can assign pulsating patterns M to revolving processes or units, and the length of the revolution can then be deduced on the basis of the repetition distance. If different revolving belts (for example steel belts) are used in a process or in a system designed to carry out a process, the repetition interval can be used to limit or assign the damaged unit (station). Different raw materials can also tend to form different patterns of damage and, if adaptive unit 23 is appropriately trained, can be useful in providing informative indication. In addition, signals S6 to S10 obtained from sensors 29 to 33 complete the effectiveness of evaluation device 15, since in this way a closed information picture can also be obtained when the described procedure is carried out. Evaluation unit 15 can compare foreign objects 10 detected by the at least two sensors 6, 7 and, in particular, patterns M of foreign objects 10 occurring over a certain period of time with signals from the causative and/or processing units (stations) of a (production) system for manufacturing manufactured boards. Two approximately square foreign objects 10 can be recognized, which in FIG. 3 are associated with a pulsating pattern M of identical particles and which in this case are intended to consist of a metal and are therefore to be removed before reaching press 2. The same applies to foreign object 10 positioned between the aforementioned objects, and which is elongated in shape and directed diagonally downwards, although this is not made of metal, but represents a piece of a sealing lip consisting of hard rubber or plastic. However, because of its size and/or location (relatively high up in mat 5), press 2, in particular belts 18a and 18b, can be endangered by these foreign objects. To be able to consider the temporal distance between detected foreign objects 10, evaluation device 15 also receives information from a conveyor belt speedometer 19 with signal line 4 via a distance/time detection device 13. Dark foreign object 10 progressing along the left edge of the enlarged area of the mat is formed by a textile part and is harmless. Evaluation device 15 will not issue an order to reject the affected section of the mat.

### COMPONENT IDENTIFICATION LISTING

| | |
|---|---|
| 1 | Production line for the production of a manufactured board |
| 2 | (Infeed into) continuous press (device/station) |
| 3 | (End of) spreading station (device/station) |
| 4 | Conveyor Device, Conveyor Belt (device/station) |
| 5 | Mat (cutout only) |
| 6 | First sensor device |
| 6a | Detection unit (for phase shift) |
| 7 | Second sensor device |
| 8 | Thickness, density or basis weight measuring device |
| 9 | Diagonal saw |
| 10 | Foreign object |
| 11 | Foreign object removal device |
| 12 | Movable cover or conveyor belt section |
| 13 | Distance/time recording device |
| 14 | Housing of the sensor devices |
| 15 | Evaluation devive |
| 16 | Deflection roller |
| 17 | Support structure of the conveyor system |
| 18a | Upper press belt |
| 18b | Bottom press belt |
| 19 | Belt speedometer |
| 20 | Displacement motor |
| 21 | Device for the detection of foreign objects |
| 22 | Measuring area |
| 23 | Adaptive unit |
| 24 | Comminution (device/station) |
| 25 | Grinding (device/Station) |
| 26 | Cooking (device/Station) |
| 27 | Drying (device/station) |
| 28 | Gluing (device/station) |
| 29 | Sensor |
| 30 | Sensor |
| 31 | Sensor |
| 32 | Sensor |
| 33 | Sensor |
| S1 | Signal (line) first sensor device |
| S2 | Signal (line) second sensor device |
| S3 | Signal (line) thickness gauge |
| S4 | Signal (line) Conveyor belt speed |
| S5 | Signal (line) to the foreign object removal device |
| S6 | Signal (line) comminution |
| S7 | Signal (line) grinding |
| S8 | Signal (line) cooking |
| S9 | Signal (line) drying |
| S10 | Signal (line) gluing |
| I | Identification |
| M | Pattern |
| SAT | Analysis of the damage history |
| SB | Damage image |
| SV | Damage prediction |

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A device for detecting at least one foreign object in a mat spread on a conveyor device in a process for producing a plurality of manufactured boards, the mat including (i) a plurality of fibers or a plurality of chips and (ii) at least one binding agent which are pressed under an increased temperature, the device comprising:
at least one first sensor device, which is configured for operating with a plurality of electromagnetic waves, a plurality of ultrasonic waves, a plurality of magnetic fields, or radiometry, for being directed at at least one spatial measuring area of the mat in order to detect the at least one foreign object, and for recognizing a material of the at least one foreign object; and
at least one second sensor device, which is configured for being aligned with the at least one spatial measuring area and for determining at least one foreign object dimension of the at least one foreign object.

2. The device according to claim 1, wherein the at least one first sensor device is configured for detecting a metal content in the material of the at least one foreign object.

3. The device according to claim 1, wherein the at least one second sensor device is configured for detecting a plurality of dimensions of the material of the at least one foreign object three-dimensionally.

4. The device according to one of the claim 1, wherein the at least one first sensor device includes a metal detector.

5. The device according to claim 1, wherein the at least one second sensor device includes a sensor that is based on a plurality of X-rays.

6. The device according to claim 1, wherein the at least one first sensor device or the at least one second sensor device includes a sensor based on thermal imaging.

7. The device according to claim 1, wherein the at least one first sensor device or the at least one second sensor device includes a sensor that is based on the plurality of ultrasonic waves and that includes a sound reflection receiver.

8. The device according to claim 1, further including an evaluation device, which is configured for performing a sensor fusion of the at least one first sensor device and the at least one second sensor device.

9. The device according to claim 8, further including a foreign object removal device which is configured for being activated depending upon a result of the sensor fusion in the evaluation device.

10. The device according to claim 8, further including a measuring device for the mat, the measuring device being configured for measuring a thickness, a density, or a basis weight of the mat.

11. The device according to claim 8, further including a distance/time detection device configured for synchronizing a plurality of sensor signals of the at least one first sensor device and the at least one second sensor device.

12. The device according to claim 8, further including a housing, wherein the at least one first sensor device and the at least one second sensor device are accommodated in the housing.

13. The device according to claim 8, wherein the evaluation device is configured for analyzing a damage history analysis.

14. The device according to claim 8, wherein the evaluation device is configured for a damage prediction.

15. The device according to claim 8, further including an adaptive unit (i) which the evaluation device includes or (ii) with which the evaluation device is operatively connected.

16. The device according to claim 15, wherein the adaptive unit is configured for identifying a plurality of at least possible damage images based on a plurality of patterns of a plurality of detected signals of a plurality of individual sensors.

17. The device according to claim 8, wherein the device is configured for being operatively connected with at least one sensor assigned to a device/station of a production line for producing the plurality of manufactured boards.

18. The device according to claim 1, wherein the device is configured for being a part of a production line which is for producing the plurality of manufactured boards and which includes a spreading station—for spreading the mat—and a continuous press—for compacting the mat—and the device which is between the spreading station and the continuous press.

19. A method for detecting at least one foreign object in a mat, the method comprising the steps of:
providing that the mat is spread on a conveyor device in a process for producing a plurality of manufactured boards, the mat including (i) a plurality of fibers or a plurality of chips and (ii) at least one binding agent which are pressed under an increased temperature in a continuous press;
using at least one first sensor device, which operates with a plurality of electromagnetic waves, a plurality of ultrasonic waves, a plurality of magnetic fields, or radiometry and which is directed at at least one spatial measuring area of the mat in order to detect the at least one foreign object, the at least one first sensor device recognizing a material of the at least one foreign object;
aligning—if necessary, temporally offset—at least one second sensor device with the at least one spatial measuring area; and
determining, by the at least one second sensor device, at least one foreign object dimension of the at least one foreign object.

20. The method according to claim 19, wherein the at least one first sensor device is configured for detecting a metal content in the material of the at least one foreign object.

21. The method according to claim 19, wherein the at least one second sensor device detects a plurality of dimensions of the material of the at least one foreign object three-dimensionally.

22. The method according to claim 19, wherein an evaluation device performs a sensor fusion of the at least one first sensor device and the at least one second sensor device.

23. The method according to claim 22, wherein, depending on a result of the sensor fusion in the evaluation device, a determination is made as to whether to activate a foreign object removal device.

24. The method according to claim 23, wherein, depending on a mat thickness or a mat density, (i) the evaluation unit issues an alert regarding a harmful foreign object or (ii) the foreign object removal device is activated.

25. The method according to claim 22, wherein the at least one first sensor device and the at least one second sensor device use different physical operating methods.

26. The method according to claim 22, wherein a distance/time detection device is used to synchronize a plurality of signals for a same one of the at least one spatial measuring area.

27. The method according to claim 26, wherein the at least one first sensor device and the at least one second sensor device simultaneously capture the same one of the at least one spatial measuring area.

28. The method according to claim 22, wherein a damage history analysis is carried out by way of the evaluation device.

29. The method according to claim 22, wherein a damage prediction is carried out by way of the evaluation device.

30. The method according to claim 22, wherein an adaptive unit associated with the evaluation device or operatively in connection with the evaluation device is supplied with a plurality of signals from a plurality of sensors, which includes the at least one first sensor device and the at least one second sensor device.

31. The method according to claim 30, wherein the adaptive unit (23) is used for an identification of a plurality of at least possible damage images based on a plurality of patterns of a plurality of detected signals of a plurality of individual sensors.

32. The method according to claim 19, wherein the method is carried out by using a plurality of signals, at least temporarily, from at least one sensor assigned to a device/station of a production line for producing the plurality of manufactured boards.

33. A manufacturing process for producing a plurality of manufactured boards, the manufacturing process comprising the steps of:
spreading, in a spreading station, a mat on a conveyor device, the mat including (i) a plurality of fibers or a plurality of chips and (ii) at least one binding agent;
compacting the mat in a continuous press under an increased temperature;
applying, between the spreading station and the continuous press, a method for detecting at least one foreign object in the mat, the method including the steps of:
using at least one first sensor device, which operates with a plurality of electromagnetic waves, a plurality of ultrasonic waves, a plurality of magnetic fields, or radiometry and which is directed at at least one spatial measuring area of the mat in order to detect the at least one foreign object, the at least one first sensor device recognizing a material of the at least one foreign object;
aligning—if necessary, temporally offset—at least one second sensor device with the at least one spatial measuring area; and
determining, by the at least one second sensor device, at least one foreign object dimension of the at least one foreign object.
